(19) United States
(12) Patent Application Publication
(10) Pub. No.: US 2009/0071478 A1
(43) Pub. Date: Mar. 19, 2009

(54) VENTILATOR

(75) Inventor: Ziv Kalfon, Kadima (IL)

Correspondence Address: HOFFMANN & BARON, LLP, 6900 JERICHO TURNPIKE, SYOSSET, NY, 11791, US

(73) Assignee: GENERAL ELECTRIC COMPANY, Schenectady, NY (US)

(21) Appl. No.: 12/212,099

(22) Filed: Sep. 17, 2008

Related U.S. Application Data
(60) Provisional application No. 60/973,019, filed on Sep. 17, 2007.

(51) Int. Cl. A61M 16/20; A62B 9/02; A61M 16/00

(52) U.S. Cl. 128/204.17; 128/205.24; 128/205.12

(57) ABSTRACT

A ventilator to replace or supplement a patient's breathing includes a control valve in the form of a proportional obstacle valve (POV) to provide improved air flow control and ventilator operation reliability. The POV includes an inlet, an outlet and a bypass. A stopcock controlled by a stepper motor directs the flow of air through the bypass and outlet permitting the turbine to operate a constant RPM yet allowing control of the airflow to a patient. The ventilator also includes inhalation and exhalation valve assemblies which improve air flow control and are easy to manufacture. The inhalation valve includes an orifice disk to allow pressure sensors to move accurately measure air flow. The exhalation valve assembly includes wings to reduce turbulence and enhance sensor accuracy. The exhalation valve assembly is arranged to have warm air from cooling the turbine blow over the assembly to reduce the possibility of condensation forming therein. The ventilator also includes an improved power supply with redundant sources of power.

This application claims the benefit of U.S. Provisional Application Ser. No. 60/973,019 filed on Sep. 17, 2007, the disclosure of which is incorporated herein by reference.

### BACKGROUND OF THE INVENTION

The present invention generally relates to a ventilator for medical use. More particularly, the invention is directed to a medical ventilator having improved airflow control, airflow sensing, increased reliability and a redundant power supply system.

A mechanical ventilator is a machine used to replace or supplement the natural function of breathing. One such device is classified as a positive pressure ventilator, meaning that air is forced out of the ventilator through a drive mechanism such as a piston, turbine, bellows, or high gas pressure. This action raises the pressure in the airways relative to atmospheric pressure, and the resulting increase in intrapulmonary pressure forces the lungs to expand. Thus, ventilators can provide continuous or intermittent mechanical ventilation to support both invasive and non-invasive needs. The ventilation is typically generated by a turbine, driven by a motor which provides the airflow and pressure.

In order to control the ventilation process, the air pressure and velocity need to be measured both during the patient inhalation and exhalation. The present invention provides an improved flow sensor mechanism to control the ventilation process. Furthermore, to ventilate at a preset pressure and flow, the air pressure and volumetric flow rate that are delivered to the patient have to be controlled. The present invention provides a mechanism that provides for improved airflow control.

Additionally, as air is drawn into the ventilator it generally passes through a filter to remove impurities. As the filter becomes obstructed with debris, the operation of the ventilator deteriorates and may eventually malfunction. The present invention provides a method to determine when the filter needs replacement.

Furthermore, the flow sensors associated with ventilators can be adversely affected by moisture. Particularly, when a patient exhales the air that is exhaled contains a high amount of humidity. If the exhaled air comes in contact with a cool surface, such as the exhalation valve and flow sensor associated therewith to measure exhaled volume, the moisture condenses and interferes with the function of the flow sensor, and in some instances, the exhalation valve. The present invention provides a means for reducing the affects of high humidity exhaled air on the operation of the sensors and valves.

Lastly, ambulatory ventilators generally include both an internal and external power source in the form of a rechargeable battery and a power cord, respectively. If the battery requires replacement, it is necessary to remove all power from the ventilator to install a new battery. Upon installation, the ventilator must be rebooted prior to operation. The present invention provides a power system which overcomes the problems associated with replacement of batteries in prior ambulatory ventilators.

### SUMMARY OF THE INVENTION

The ventilator formed in accordance with the present invention overcomes each of the shortcomings discussed above with respect to operator control, reliability and feedback from the patient. The ventilator of the present invention includes a turbine for generating a positive pressure airflow. The ventilator further includes a control valve in the form of a proportional obstacle valve which is driven by a stepper motor. The proportional obstacle valve includes a stopcock rotatably mounted in the valve to control the flow of air therethrough. The control valve includes an inlet, an outlet and a bypass passageway such that operation of the proportional obstacle valve controls the flow of air from the inlet through the bypass passageway and outlet. The ventilator further includes a means for directing airflow from the control valve outlet to the patient. The directing means typically includes flexible tubing and a mask attachable to the patient's nose and mouth. Preferably, the turbine operates at an optimal RPM for energy efficiency and the proportional obstacle valve controls the airflow to the patient by directing air through both the bypass passageway and outlet. Furthermore, the proportional obstacle valve includes a stopcock rotatably movable by the motor, the stopcock being in close proximity to but not in contact with the opening in which the stopcock rotates.

The airflow directing means preferably also includes an inhalation strut assembly and exhalation valve assembly. The inhalation strut assembly may include an area of reduced diameter in the form of an orifice disk to provide a pressure differential on the inlet and outlet sides thereof. The inhalation strut assembly includes at least one pressure sensor positioned to receive input from both an inlet and outlet side of the orifice disk. Additionally, the outlet side of the inhalation strut preferably includes a diffuser to increase the dynamic range of differential pressure for greater sensor sensitivity.

The exhalation valve assembly has a series of sensors associated therewith. In the preferred embodiment, the exhalation valve assembly includes an area of reduced diameter between the inlet and outlet, the area of reduced diameter including a plurality of wings extending radially inwardly to reduce airflow turbulence as air passes therethrough. A sensor is provided to receive input from openings in the area of reduced diameter and the outlet portion of the exhalation valve assembly. Preferably, both the exhalation valve assembly strut and inhalation valve assembly strut are constructed as a one-piece injection molded component to improve manufacturability and reduce costs. These components are easily removable from the unit for sterilization and replacement.

The ventilator formed in accordance with the present invention also includes an air inlet in the housing thereof and an inlet air filter associated therewith. The ventilator is also provided with a means for determining and indicating to a user that the air inlet filter needs replacement. Preferably, the ventilator is provided with a sensor positioned downstream of the air inlet and upstream of the turbine air inlet. Should the air inlet filter become clogged, a vacuum would be created which is sensed by the sensor to indicate the filter needs replacement.

The ventilator also preferably includes a means for directing heated air to flow over the exhalation valve assembly. In one embodiment, the ventilator includes a fan for cooling the turbine. The cooling air which is heated by the turbine is directed to flow over the exhalation valve assembly to warm the assembly. In another preferred embodiment, the turbine assembly which includes a turbine and drive motor, is provided with an internal heat sink. The turbine drives air over the heat sink and a portion of the air heated by the turbine is directed to flow over and warm the exhalation valve assembly. The warming of the exhalation valve assembly reduces the probability of the formation of condensation from the high humidity air exhaled by the patient.

The ventilator of the present invention also preferably includes a redundant power supply system such that rebooting of the unit is not necessary upon switching among the power supplies. Preferably, the unit includes an external AC power cord, an internal rechargeable battery, an external battery adaptable to be plugged into the ventilator and an internal backup battery. The unit further includes a power switching system which selects the appropriate power source.

### BRIEF DESCRIPTION OF THE DRAWINGS

FIG. 1 is a perspective view of a ventilator formed in accordance with the present invention.

FIG. 2 is an illustration of the pneumatic box unit of the ventilator formed in accordance with the present invention.

FIG. 3 is a pneumatic block diagram of the pneumatic components of the ventilator formed in accordance with the present invention.

FIG. 4 is a cross-sectional view of the proportional obstacle valve (POV) formed in accordance with the present invention in a fully open state.

FIG. 5 is a cross-sectional view of the POV of FIG. 4 in a closed state.

FIG. 6 is a cross-sectional view of an inhalation strut formed in accordance with the present invention.

FIGS. 7 is a top plan view of the inhalation strut illustrated in FIG. 6.

FIG. 8 is a cross-sectional view of an exhalation valve and strut formed in accordance with the present invention.

FIG. 9 is an expanded cross-sectional view of the exhalation valve illustrated in FIG. 8.

FIG. 10 is a block diagram illustrating the inlet filter sensor formed in accordance with the present invention.

FIG. 11 is a block diagram illustrating a redundant power supply system formed in accordance with the present invention.

### DETAILED DESCRIPTION OF THE PREFERRED EMBODIMENTS

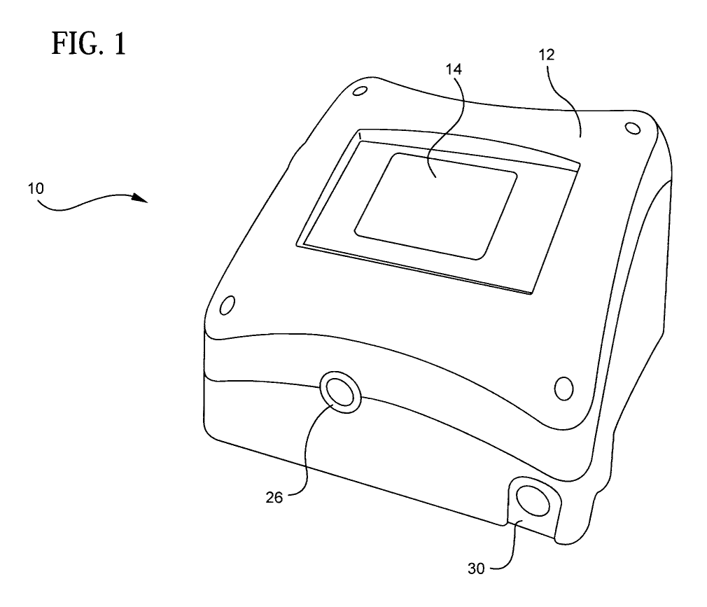

A medical ventilator formed in accordance with the present invention is illustrated in FIG. 1. The ventilator 10 includes a housing 12 with a touch screen 14 to control the operation of the ventilator, provide patient information, and provide feedback from sensors to monitor a patient's breathing. Also shown in FIG. 1 is the inhalation valve assembly 26 and exhalation valve assembly 30 which, through use of tubing (not shown) to the patient, places the medical ventilator in fluid communication with the patent.

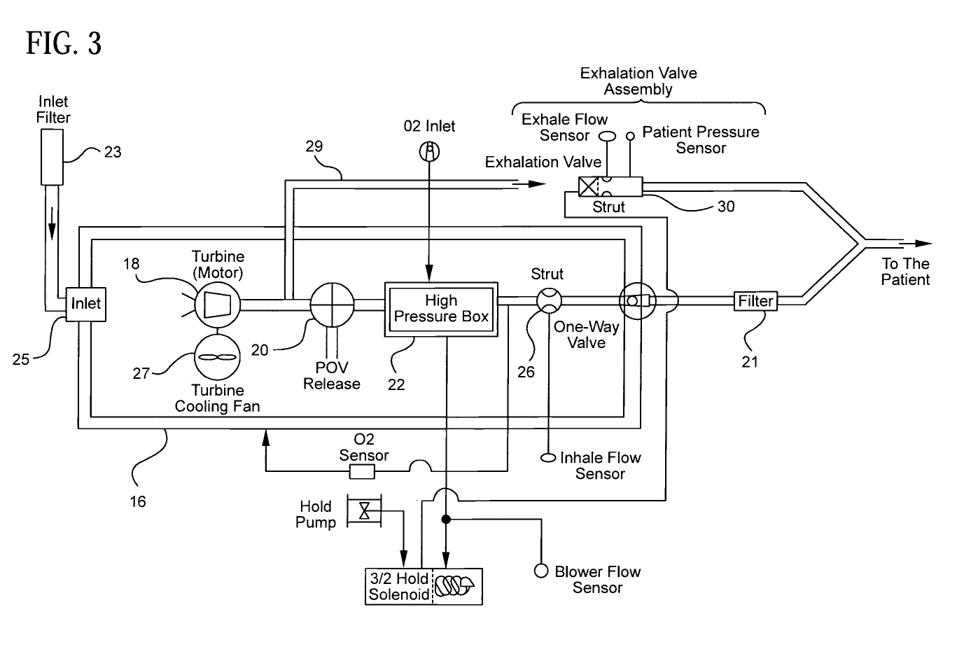

FIG. 3 is a pneumatic block diagram of the pneumatic components of the ventilator. The major pneumatic components includes a turbine assembly 18 including a turbine and drive motor to create a positive air flow, a control valve to control air flow in the form of a proportional obstacle valve (POV) 20 having a movable valve operated by a stepper motor 33 coupled to the POV, a high pressure box 22, an inhalation valve and strut 26 to provide a one-way path for airflow to a patient, an exhalation valve and strut 30 to receive exhaled air for patient monitoring and a plurality of pressure/flow sensors.

The airflow path to the patient preferably includes an air filter 21. The ventilator draws ambient air into the device through an inlet filter 23 in fluid communication with an inlet 25 coupled to the turbine intake. Thus, air provided to a patient is filtered upon entry into the ventilator as well as prior to being output to the patient.

In a preferred embodiment, to cool the turbine during operation, the turbine assembly 18 is provided with an internal heat sink. Alternatively, a cooling fan 27 may be used to blow air over the turbine assembly. As will be discussed in great detail below, the air heated by the heat sink or the cooling fan is directed to flow over and warm the exhalation valve assembly 30. (See air flow path 29 in FIG. 3).

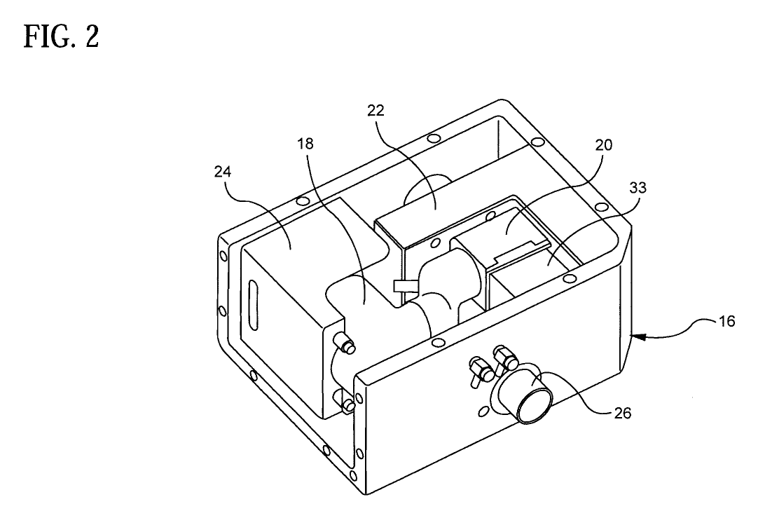

FIG. 2 illustrates the pneumatic box unit 16 of the ventilator 10. The major components of the pneumatic box unit 16 are the turbine 18 which is driven by a motor, proportional obstacle valve (POV) 20 used to regulate flow of air to the patient, a high pressure box 22, a noise damper 24 and a one-way inhalation valve and strut 26.

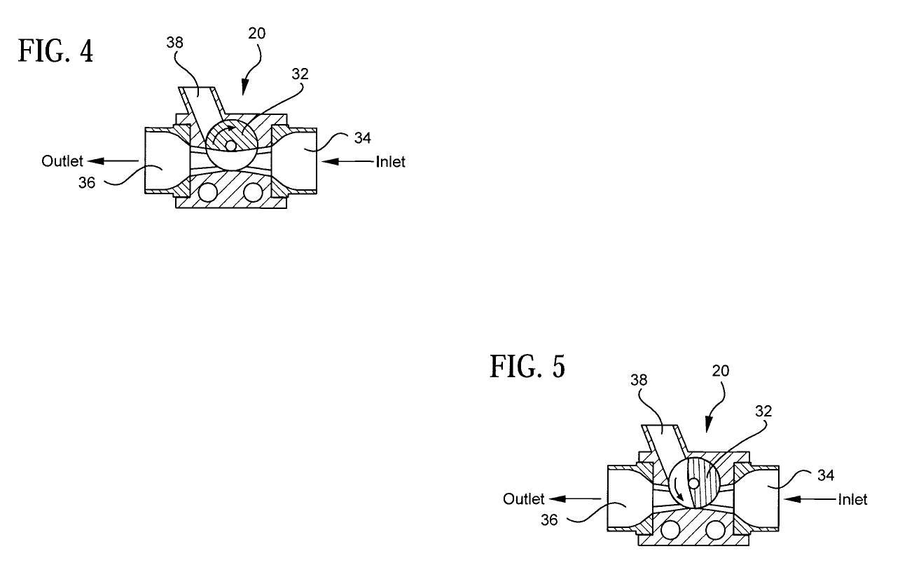

In order to provide improved airflow control to the patient and ventilator operation reliability, the present invention adopts the use of the proportional obstacle valve (POV) 20 as shown in FIGS. 4 and 5. The POV 20 includes a stopcock 32 driven by a stepper motor 33 (FIG. 3) and provides very low flow resistance.

The POV 20 works like a faucet with two outlets. As shown in FIG. 4, the air from the turbine enters the POV via the main inlet 34. Turning the stopcock 32 controls the area of the passageways forming the outlets. The wider outlet 36 delivers the air to the patient, while the narrower outlet is a bypass 38 that returns the surplus air to the turbine inlet. By manipulating the open area of both outlets through rotation of the stopcock 32, the user can precisely control the amount of air delivered to the patient. As shown in FIG. 4, the stopcock 32 is in its fully open state with no air being directed to the bypass 38. FIG. 5 illustrates the stopcock 32 in its fully closed state.

The POV 20 is highly reliable and can operate continuously for millions of cycles. The stopcock 32 can operate without a reduction in speed or impermeability. Furthermore, the stopcock 32 can accelerate rapidly. For example, the stopcock can transition from its closed to open state in approximately 30 msec. At the same time, the stopcock engine, i.e., stepper motor 33, is small and energy efficient and configured for battery-operation. Moreover, the bypass arrangement allows the speed of the turbine to be kept high rather than modulating the RPM's of the turbine to control flow which consumes unnecessary power. By varying the amount of air directed to the bypass using the stopcock 32, flow to the patient is controlled without modifying the turbine speed. Accordingly, the turbine may be operated at an optimal RPM for maximum energy efficiency with the flow of air to the patient being controlled by the POV 20. Thus, the POV 20 provides an infinitely variable bypass for improved ventilator control.

The improved airflow control of the present invention using the POV 20 is based on the following two principles: use of a bypass in the airway passage; and use of air for impermeability or sealing. The use of a bypass 38 as part of the POV air passage, where the surplus air is released instead of being delivered to the patient, provides immediate control over the delivered pressure. The bypass 38 also enables much better control over the volumetric flow rate delivered to the patient by providing controlled release of the turbine volumetric flow rate.

Efficiency of operation of the ventilator device is important, in general, and especially in a portable ventilator operating by battery power. The POV operation provides the patient with the high pressure air flow from the turbine when the stopcock 32 is in open position, with the smallest losses due to air leakage. Additionally, unnecessary load on the stopcock motor 33 is prevented, by providing a small gap between the stopcock 32 and valve body thereby reducing the friction on the stopcock as will be discussed in greater detail below. The reduction in friction also meets the requirement for high reliability which prevents any solution that causes increased wear on components which could lead to system failure.

However, this impermeability of the pneumatic unit using a POV cannot be based upon friction, as in a regular faucet, for the following reasons: the engine or motor load would increase as the engine would have to overcome the component's friction along with the stopcock inertia, which occurs when the stopcock changes its position; the components would wear out more quickly, and, as a result, reduce the impermeability efficiency; the mechanism would be more costly, since it would require specific materials, detailed design and more accurate manufacture. Instead of friction, the POV of the present invention uses air to make the air passage impermeable.

Any fluid, including air, has a viscosity that causes friction and shear forces. When a fluid passes through a tube, there is a layer in the immediate vicinity of the bounding surface that does not flow. This layer is called the boundary layer. This layer affects the adjacent layer with shear forces, causing the neighboring layer to decrease its speed. This process repeats itself with each layer of the fluid, until the shear force is decreased to the point where it does not affect the flow. The number of layers with different velocities has a direct proportion to the viscosity values.

The POV 20 of the present is based on the border layer principle described above. To apply this principle in the POV, the diameter of the stopcock 32 is approximately 0.1 mm less than the diameter of the opening in which it rotates. This difference in diameter of the POV prevents friction between the stopcock and the valve cylinder. In addition, the solution of the present invention allows some tolerance towards inaccuracy during manufacture. However, this slight difference in diameter combined with a unique air passage geometry permits only a few boundary layers, which are not sufficient for the flow to overcome the shear forces. Impermeability is thus created without friction. While those skilled in the art will appreciate that the impermeability is not absolute, any leakage is reduced to negligible values which do not adversely affect operation of the ventilator. Furthermore, those skilled in the art will understand that the tolerances and measurements identified above are for illustrative purposes and may be modified without departing from the scope and spirit of the invention.

Another aspect of the present invention is a flow meter mechanism in the form of inhalation/exhalation strut assemblies which provide for improved flow sensor measurements. The exhalation valve assembly includes a valve system which is user serviceable for easy replacement. Both the inhalation and exhalation strut assemblies are made from molded plastic for ease of manufacture and to reduce cost. The flow sensor for the inhalation strut assembly is based upon the use of an orifice disk with an aperture and a diffuser while the exhalation valve assembly flow sensor is based upon a diffuser with wings to stabilize flow and reduce turbulence.

An orifice flow meter disk uses the same principle as a Venturi nozzle, i.e., it is based on Bernoulli's principle which holds that a slow-moving fluid exerts more pressure than a fast-moving fluid. The orifice flow meter disk is a disk with an aperture in the middle. This disk is placed perpendicular to the fluid flow direction (pipe axes), which forces the fluid to flow from a wide passageway or tube through the smaller aperture. The fluid mean velocity then increases to compensate for the reduction in the tube area (assuming incompressible fluid behavior at subsonic velocities, such as air at the device's functional flow rate settings). The actual cross-sectional area of the rapid mean velocity is less than the area of the aperture, due to inverse fluid flow and is called vena contracta, which is located at a point where the fluid flow begins to diverge after passing through the aperture.

As the fluid continues to flow through the tube, the tube area returns to its original size, and the fluid velocity returns to original velocity. The pressure increases, but it does not return to its original value due to energy losses known as head loss.

By measuring the fluid static pressure in front of and immediately after the disk, at the assumed vena contracta as discussed above, flow rate can be calculated. Alternatively, the secondary flow rate inhibited by static pressure differences between measurement ports can be measured for the purpose of flow rate assessment.

A subsonic diffuser may be used for conversion of kinetic energy of a fluid into enthalpy or static pressure, assuming the fluid is incompressible (air at the device's functional flow rate settings). A subsonic diffuser consists of a tube which expands in diameter as air flows downstream. The cross-sectional area of the tube expands without any change in volumetric flow rate of the fluid in accordance with the law of conservation of mass. Thus, a mean velocity decrease in direct proportion to the area expansion of the tube is accomplished which can be measured and used to control the ventilator.

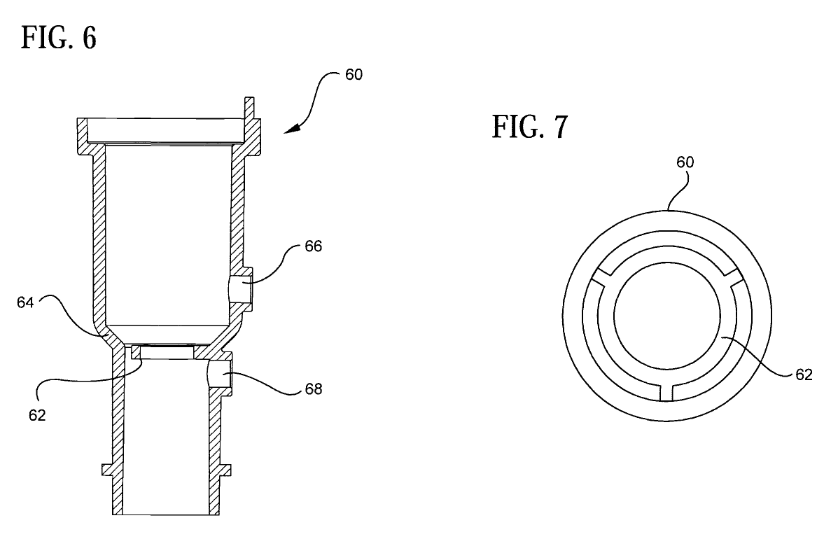

The present invention includes an inhalation strut assembly 60 that enables measurement of the air static pressure or its induced secondary flow rate and may measure other fluids as well (liquid and gas). As shown in FIGS. 6 and 7, the inhalation strut 60 operates by geometrically manipulating the air passages to create a pressure drop that is dependent on the fluid's velocity. This dependency can be calculated and calibrated in order to translate the pressure drop into velocity.

The inhalation strut 60 provides accurate velocity measurements, from zero volumetric flow rate up to 200 L/min. It also provides differential pressure ranging from 0 to 5 mBar, respectively and close to linear relation between the pressure drop and the volumetric flow rate. Due to its design, the inhalation strut assembly can be manufactured as one component by plastic injection molding technique, thereby reducing the manufacturing costs. Not only is the integrally molded strut easier and less expensive to manufacture, but it is also simple to replace in the ventilator, if necessary.

The inhalation strut 60 is unique in its geometry combining an orifice disk 62 and a degenerated diffuser 64. The orifice disk 62, like a Venturi nozzle, causes energy losses that are reflected in pressure drop measurements (i.e. head loss, mainly at low velocities). The disk of the present invention may be grooved to increase measurement sensitivity at low flow rates. As can be seen through the governing equation,

ΔP=KQ²

the pressure decreases rapidly as the velocity increases. The sensor is required to measure these rapid changes of pressure over the functional full flow rate range, without orifice sensitivity deficiency at high flow rates. A subsonic diffuser reduces the pressure differences at high values of volumetric flow rates with the least possible effect on the differences at low values of volumetric flow rates. As explained, the diffuser 64 reduces the flow velocity and thus increases the static pressure difference. For this reason, the inhalation strut 60 of the present invention built using diffuser geometry, compensates for the orifice effect at high flow rates by contra increasing the static pressure.

Theoretically, diffuser pressure difference behavior and orifice disk head loss behavior are negatively related. The different efficiency characteristics of the combined apparatus entail partial linearization of pressure flow relation at relatively high flow rates, while maintaining the measurement sensitivity at low flow rates. The inhalation strut 60 of the present invention combines the complementary mechanisms of the orifice disk 62 and the diffuser 64, thereby resulting in a measurement tool that can measure the flow accurately, in both high and low volumetric flow rate. To measure flow, the inhalation strut is provided with two pressure measurement ports 66, 68 coupled to a sensor. (See FIG. 2). The two ports 66, 68 form a differential pressure bridge, port 66 being positioned in the large diameter area, of the strut and port 68 being located in the smaller diameter area such that the pressure differential measured between the two ports accurately approximates flow. The inhalation strut 60 of the present invention maintains low pressure differences (5 mbar) and as previously mentioned, may be built as one component manufactured by plastic injection.

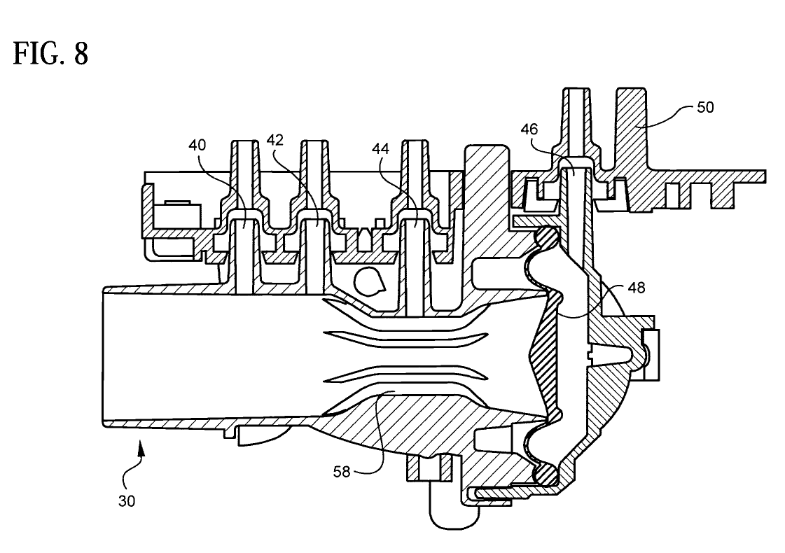

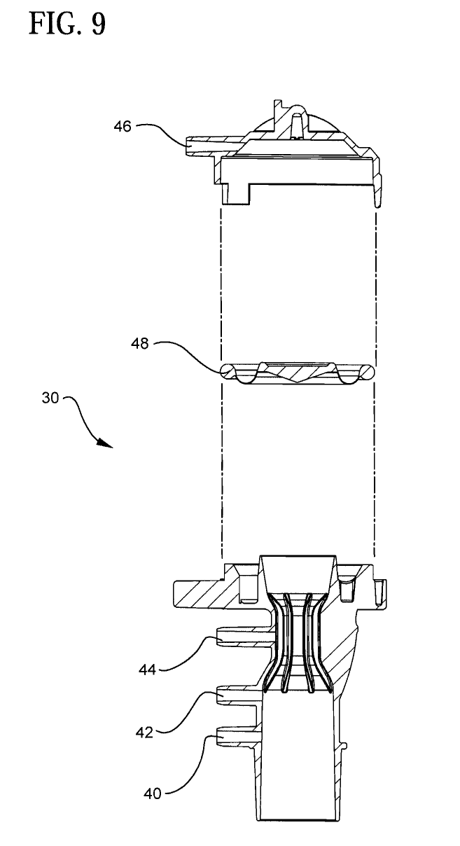

Referring to FIGS. 8 and 9, the exhalation valve and strut assembly 30 includes a patient pressure port 40 and two ports 42, 44 forming a differential pressure bridge, port 42 being positioned in an area of the valve which is larger in diameter than that of port 44. A pressure sensor is provided with respect to port 40 for patient pressure sensing and another sensor is provided for the differential pressure bridge 42, 44 as an exhale flow sensor. (See FIG. 3). The sensed pressure differential between ports 42, 44 accurately approximates exhale flow. A fourth port 46 provides pressure to operate the exhalation valve 48 which is in the form of a flexible membrane. The area of reduced diameter associated with the differential pressure bridge includes stabilizing flow wings 56 to reduce turbulence and improve sensor reliability. The flow wings 56 are arranged to extend into the passageway along a longitudinal flow axis. The wings 56 extend from the end of the large diameter inlet passageway to the beginning of the large diameter outlet passageway in the reduced diameter portion of the exhalation valve strut. The wings 56 are preferably equally spaced about the reduced diameter portion thereby reducing turbulence to enhance the accuracy of the flow sensors. FIG. 9 illustrates an exploded view of the exhalation valve and strut assembly 30.

The exhalation valve and strut assembly 30 is removably coupled to a manifold 50 which connects the assembly into the ventilator housing. The exhalation valve and strut assembly includes a pair of movable levers (not shown) which hold the assembly in position. The exhalation valve and strut assembly 30 can be easily removed and replaced in the manifold 50. Once removed and disassembled, the parts are autoclavable for reuse.

The ventilator of the present invention also provides a means for reducing the affects of high humidity exhaled air on the operation of the exhalation valve assembly and sensors. As a patient exhales, the exhaled air is heated by the patient's lungs and airways and contains a high amount of humidity, in some cases approaching 100%. This high humidity air travels through the exhalation valve and its associated flow sensors for measuring exhaled air volume. If the high humidity exhaled air comes in contact with a cool surface, the moisture condenses and forms condensate in the form of water droplets. This condensate can interfere with the function of the flow sensor and, in some cases, the exhalation valve. In some circumstances, droplets of condensate have formed under the ventilator.

The present invention includes a means for reducing the probability of condensate forming, which includes a means for directing heated air over the exhalation valve assembly. The turbine generates heat which can be destructive to the turbine bearings over time. To mitigate the effects of heat on the turbine bearings, as shown in FIG. 3, a fan 27 is placed adjacent the turbine assembly 18 to blow cooling air over the turbine. Alternatively, the turbine assembly may preferably include an internal heat sink located in the airflow path generated by the turbine, a portion of which is directed to flow over the exhalation valve assembly. Typically, the heated air from cooling the turbine assembly is exhausted from the unit. In the present invention, the heated air is directed to flow over the exhalation valve assembly to raise the temperature of the exhalation valve and flow sensor so that it does not become a condensation point for high humidity exhaled air from the patient. (See e.g. airflow path 29). Thus, by using the heated air from the cooling the turbine, the temperature of the exhalation valve assembly can be raised to avoid condensation from forming on those component parts. Since condensation is avoided, the exhalation valve and associated sensors do not experience the difficulties of prior art ventilators with respect to the formation of condensation. Furthermore, the design of the present invention does not add any component parts but uses the heated air which would otherwise by exhausted to the atmosphere to reduce the probability of condensate forming in and around the exhalation valve assembly and associated sensors.

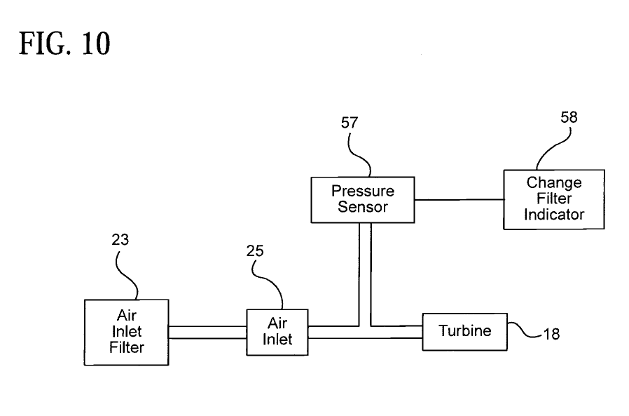

Another feature of the present invention is directed to a means for detecting and indicating to the user that the inlet air filter needs replacement. Referring to FIG. 10, air for ventilation is drawn into the machine through an inlet filter 23. Typically, the inlet filter 23 is located on the ventilator housing 12 and filters out particulars from the air delivered to the patient. For this reason, it is important to prevent any obstruction to the filter airways.

Obstructions in the inlet filter 23 will eventually cause the ventilator to deteriorate or to malfunction. It is necessary to evaluate the condition of the filter in order to notify the operator when filter replacement is required. Filter replacement, however, is dependent on the operating environment of the machine, where the amount of dust in the air may vary considerably. Thus, filter replacement cannot be scheduled as a preventive maintenance operation, since the time for replacement may vary. Instead, it is necessary to constantly check the efficiency of the filter.

The present invention overcomes this problem by providing an air inlet sensor. The sensor detects the efficiency of the filter by measuring the amount of air entering the machine. When the filter is obstructed, its resistance increases, which means that less air is drawn into the machine. Since the turbine draws air in from the air inlet entrance, a vacuum is created if not enough air enters via the filter.

As shown in FIG. 10, the present invention provides a pressure sensor 57 placed on the main electronic board of the machine, which is connected via a tube to the air entrance of the turbine. When the sensor reading reaches a preset value establishing the presence of a vacuum and hence a dirty filter, the machine prompts the operator to replace the filter by means of a service message 58 displayed on the display screen and/or via an audible signal.

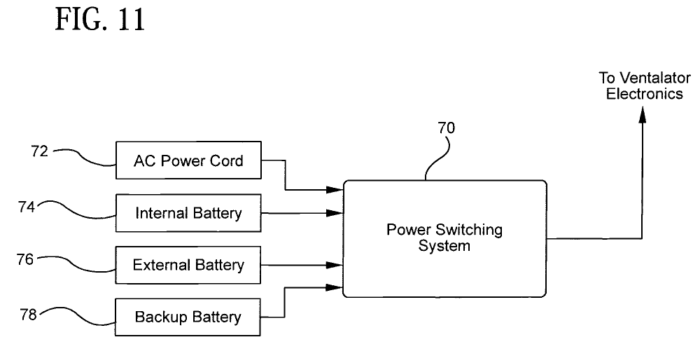

A still further feature of the present invention is an improved power source. As shown in FIG. 11, the ventilator of the present invention includes separate, redundant power sources including an external a/c power cord 72 for use when a power outlet is accessible and for charging an internal integrated battery 74. Thus, when a power outlet is not available, the ventilator may be operated with the internal integrated battery 74. The ventilator also includes an external battery 76 which may be plugged into the unit for power. Finally, the ventilator of the present invention includes a backup battery 78 should the primary source of power fail. Each of the sources is electrically coupled to a power switching system 70 which automatically selects the desired source of power to operate the ventilator. In view of the redundant power sources, a battery may be replaced without the need for the unit to be shut down and rebooted.

Although the illustrative embodiments of the present invention have been described herein with reference to the accompanying drawings, it is to be understood that the invention is not limited to those precise embodiments, and that various other changes and modifications may be effected therein by one skilled in the art without departing from the scope of the invention.

What is claimed is:

1. A ventilator for replacing or supplementing a patient's breathing, comprising:
a turbine for generating a positive pressure air flow;
a control valve comprising a proportional obstacle valve having a stopcock rotationally movable by motor, the control valve including an inlet, an outlet and a bypass passageway, the proportional obstacle valve operating to control the flow of air from the inlet through the bypass passageway and outlet; and
means for directing air flowing from the control valve outlet to the patient.

2. A ventilator as defined in claim 1, wherein the motor is a stepper motor.

3. A ventilator as defined in claim 1, wherein the stopcock of the proportional obstacle valve is in close proximity but not in contact with an opening in which the stopcock rotates.

4. A ventilator as defined in claim 1, wherein the turbine operates at an optimal RPM for energy efficiency and the proportional obstacle valve controls air flow to the patient by directing air through the bypass passageway.

5. A ventilator as defined in claim 1, wherein the air flow directing means includes an inhalation strut assembly having an area of reduced diameter in the form of an orifice disk.

6. A ventilator as defined in claim 5, wherein the inhalation strut assembly includes at least one pressure sensor positioned on the inlet and outlet side of the orifice disk.

7. A ventilator as defined in claim 6, wherein the outlet side of the inhalation strut includes a diffuser.

8. A ventilator as defined in claim 1, wherein the air flow directing means includes an exhalation valve assembly having an area of reduced diameter between an inlet and outlet, the area of reduced diameter including a plurality of wings extending radially inwardly to reduce air flow turbulence.

9. A ventilator as defined in claim 8, wherein the exhalation valve assembly includes an exhalation strut which is a one-piece, injection molded component.

10. A ventilator as defined in claim 1, further comprising a ventilator air inlet in fluid communication with an air inlet of the turbine, the ventilator air inlet including an inlet air filter, and means for determining and indicating to a user that the inlet air filter needs replacement.

11. A ventilator as defined in claim 10, wherein the determining means comprises a pressure sensor positioned in the turbine air inlet.

12. A ventilator as defined in claim 1, further comprising a redundant power supply including an internal rechargeable battery, an external power cord, an external battery adaptable to be plugged into the ventilator and an internal backup battery.

13. A ventilator as defined in claim 1, wherein the air flow directing means includes an exhalation valve assembly and the ventilator further comprises means for cooling the turbine, and wherein air heated by the turbine is directed to flow over the exhalation valve assembly to warm the assembly and thereby reduce the probability of the formation of condensation.

14. A ventilator for replacing or supplementing a patient's breathing, comprising:
means for generating a positive pressure air flow to be delivered to the patient;
means for cooling the generating means and producing an air flow of heated air;
an exhalation valve assembly for monitoring the flow of air exhaled by the patient; and
means for directing the flow of heated air over the exhalation valve assembly to warm the assembly thereby reducing the probability of condensation forming therein.

15. A ventilator as defined in claim 14, further comprising a ventilator air inlet in fluid communication with an air inlet of the turbine, the ventilator air inlet including an inlet air filter, and means for determining and indicating to a user that the inlet air filter needs replacement.

16. A ventilator as defined in claim 14, wherein the generating means comprises a turbine and further wherein the cooling means comprises one of a heat sink and cooling fan.

17. A ventilator as defined in claim 14, wherein the generating means comprises a turbine and further wherein the turbine is in fluid communication with a proportional obstacle control valve, the proportional obstacle control valve having an inlet, an outlet and a bypass passageway to control air flow output from the turbine.

18. A ventilator for replacing or supplementing a patient's breathing comprising:
means for generating an air flow to be delivered to the patient;
an inhalation strut assembly having an area of reduced diameter in the form of an orifice disk, the inhalation strut assembly including at least one pressure sensor positioned on each of the inlet and outlet side of the orifice disk; and
an exhalation valve assembly including an exhalation strut, the exhalation strut having an area of reduced diameter which includes therein a plurality of wings extending radially inwardly to reduce air flow turbulence.

19. A ventilator as defined in claim 18, wherein the air flow provided to the patient flows through a proportional obstacle control valve (POV) which includes an inlet, an outlet and a bypass passageway.

20. A ventilator as defined in claim 18, wherein the generating means includes an air inlet having an air inlet filter therein, the air inlet being in fluid communication with an inlet to a turbine, and further including a pressure sensor downstream of the air inlet filter and upstream of the turbine air inlet to sense air pressure and, upon sensing a preset value, providing an indication that the air inlet filter needs replacement.
